(19) United States
(12) Patent Application Publication
(10) Pub. No.: US 2007/0265584 A1
(43) Pub. Date: Nov. 15, 2007

(54) VENOUS PROSTHESIS AND VASCULAR GRAFT WITH ACCESS PORT

(76) Inventors: Robert O. Hickman, Edmonds, WA (US); Marc Jaker, New Brighton, MN (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/675,348

(22) Filed: Feb. 15, 2007

Related U.S. Application Data
(60) Provisional application No. 60/774,004, filed on Feb. 15, 2006.

(51) Int. Cl. A61M 39/02

(52) U.S. Cl. 604/288.01

(57) ABSTRACT

A venous polytetrafluoroethylene (PTFE) graft with integral access port system. The graft may have one, two, or more ports, which then exits the skin. The prosthesis may be made of fluoropolymer tubing fabricated in such a way as to involve several performance features. The graft may have holes for suturing, anchoring, or bio-integration to the artery. The ports have natural acting restrictions or valves at the graft juncture, throughout the implanted part body of the device and at the exit site. The ports are also naturally self-purging. A port may also incorporate a redundant hermetic hemostatic valve and closure system at the exit site.

### CROSS-REFERENCE TO RELATED APPLICATIONS

This application claims priority to and benefit of U.S. Provisional Application No. 60/774,004, entitled “Venous Prosthesis and Vascular Graft with Access Port” and filed on Feb. 15, 2006.

### FIELD OF INVENTION

The present invention relates generally to vascular grafts and percutaneous prostheses. In particular, the present invention relates to vascular grafts and percutaneous prostheses with an integral access port system for vascular access such as those that may be used with patients undergoing hemodialysis for kidney failure.

### BACKGROUND OF THE INVENTION

Vascular access ports, shunts and catheters are used for hemodialysis, transfusions, chemo-drug therapy and long-term nutritional support. These port systems have become a major part of acute and chronic care. In spite of the improvements made in catheter materials, nursing care, catheter care protocols, and bonding of drugs (e.g., antibiotics, antithrombic agents), the incidence of infection and clotting episodes is still considerable. Many of these bouts of infection and thrombosis are life-threatening or debilitating. In addition, infections and thrombosis may prolong or require additional medical and surgical care, which is extremely expensive. Approximately 12% to 15% of end stage renal failure patients will require central venous catheters for hemodialysis during some part of this treatment.

In addition, almost all oncology patients will require central venous catheters at some time during their therapy. Some patients will require tunneled, cuffed catheters and ports for a long period of time from months to even years. There are an estimated 50,000 patients or more in the United States who require daily total parental nutritional support administered via a long term indwelling central venous catheter.

The dialysis population is growing each year and currently there is a push towards daily hemodialysis. Based on clinical data from Europe and in the United States, these patients seem to enjoy an increased state of “well being”. If indeed the American dialysis population turns to daily dialysis, there will be an even greater need to facilitate vascular access by; (1) introducing a “needle-less” vascular access graft (making blood access more patient friendly); (2) by increasing and improving the quality and performance of the current expanded polytetrafluoroethylene (PTFE) shunts; and (3) by reducing or eliminating the need for long-term permanent indwelling dialysis catheters.

Additionally, some patients have vascular access through an implanted port system. These are typically made of titanium, and have a body with a silicone encapsulated support screen, allowing access to a “reservoir”. This system still requires needle access and is also prone to protein disposition clotting, thrombosis, and the like.

The present invention addresses these long felt needs with a new and improved venous prosthesis and vascular graft with access port.

### SUMMARY OF THE INVENTION

The present invention permits easy and rapid placement, or replacement, of a variety of long and short-term catheters. Any cannula or catheter placed via this novel device can remain in place for the duration of any specific treatment such as four hours hemodialysis or eight hours infusion of total parental nutrition or a course of IV chemotherapy. To the extent that the cannula or catheter is easily removed, this will eliminate the need for long-term indwelling central venous catheters. The inserted device may also be accessed for routine withdrawal of blood for tests.

One result will be considerable reduction in the complications (infection and thrombic episodes) currently associated with long-term indwelling central venous catheters.

The present invention, among other things, eliminates the need for needles and their risk (e.g., needle sticks to care givers), reduces tissue damage and scarring in the patient, and also reduces infection rates. The present invention uses a non-contaminating probe system that incorporates fluoropolymer in its unique inverting sheath design.

The present invention provides arterio-venous access to a major vein such as the internal jugular vein, subclavian vein, femoral vein and femoral artery, by way of a unique polymer film graft which has one, two, or more ports that communicate through the patient's skin.

### BRIEF DESCRIPTION THE DRAWINGS

A more complete understanding of the present invention may be derived by referring to the detailed description and claims when considered in connection with the drawing Figures, where like reference numbers refer to similar elements throughout the Figures, and:

FIG. 1 illustrates a vascular graft in accordance with an embodiment of the present invention;

FIG. 2 illustrates a vascular graft in a stage of manufacturing in accordance with an embodiment of the present invention;

FIG. 3 illustrates a tensilized vascular graft in accordance with an embodiment of the present invention;

FIG. 4 illustrates access ports in accordance with an embodiment of the present invention;

FIG. 5 illustrates tensilized access ports in accordance with an embodiment of the present invention;

FIGS. 6 and 7 illustrate access ports attached to a vascular graft in accordance with an embodiment of the present invention;

FIG. 8 illustrates an access port attached to a vascular graft in accordance with an alternative embodiment of the present invention;

FIG. 9 illustrates an access port attached to a vascular graft in accordance with an embodiment of the present invention;

FIG. 10 illustrates access ports attached to a vascular graft in accordance with another embodiment of the present invention;

FIGS. 11 and 12 illustrate a cannula accessing a vascular graft via an access port in accordance with an embodiment of the present invention;

FIGS. 13-17 illustrate a valve assembly for use with a vascular graft in accordance with an embodiment of the present invention; and

FIG. 18 illustrates a vascular graft in accordance with an embodiment of the present invention.

### DETAILED DESCRIPTION

The present invention may be described herein in terms of various hardware components and modules and processing steps. It should be appreciated that such modules and steps may be realized by any number of hardware components configured to perform the specified functions. For example, the present invention may employ various shaped tubes, sheaths, and the like, which may carry out a variety of functions. In addition, those skilled in the art will appreciate that the present invention may be practiced in any number of contexts and that the illustrative embodiment as described herein is merely one exemplary application for the invention. For example, the present invention may be applicable to various types of animals and other applications that require the use of various types of vascular grafts. Further, such general techniques that may be known to those skilled in the art are not described in detail herein.

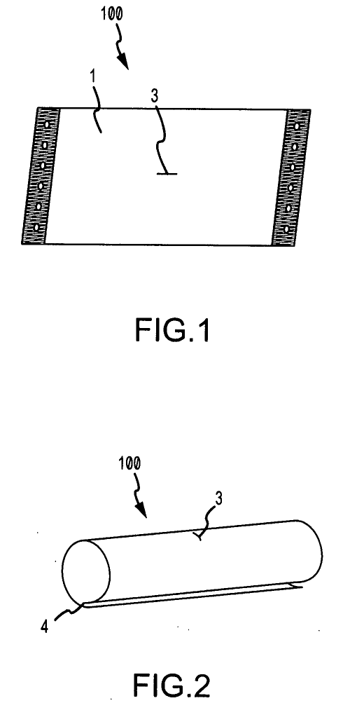

With reference to FIG. 1, in accordance with an embodiment of the present invention, a vascular graft 100 may be formed from a polytetrafluoroethylene (PTFE) sheet 1 having less than 0.010″ thickness. PTFE sheet 1 may or may not have pre-formed holes 2 for suturing to a blood vessel. The sheet includes one or more pre-slots 3 through which “port”(s) may later be welded.

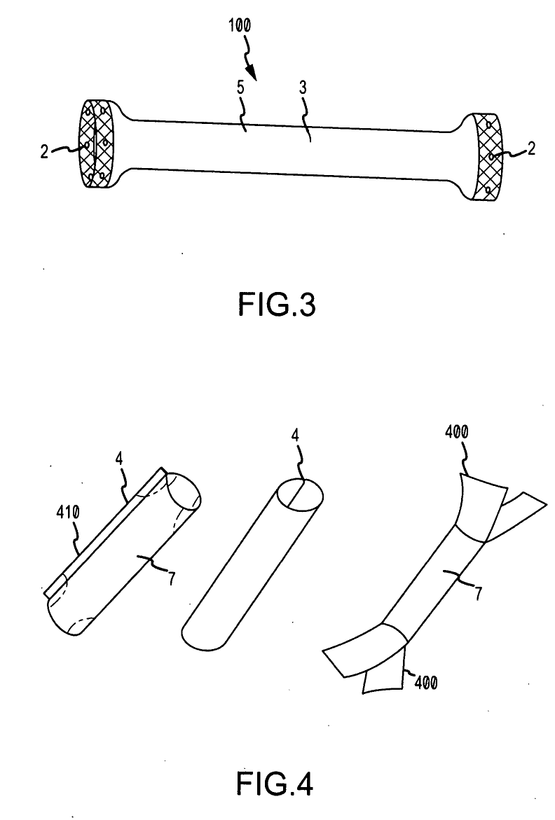

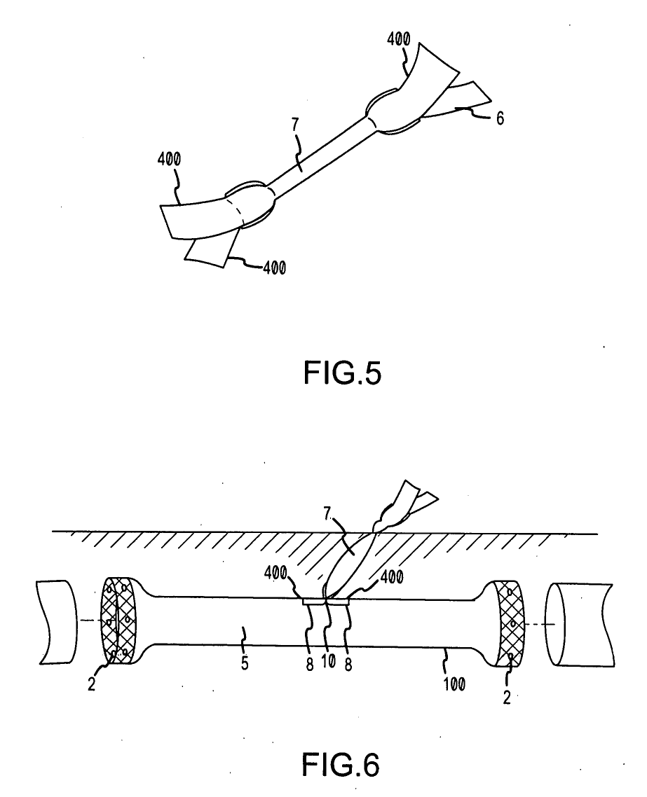

In accordance with an embodiment of the present invention, FIG. 2 illustrates thin polymer sheet 1 with the sides overlapped. With reference to FIG. 3, the main graft body center section 5 is illustrated. As shown, graft 100 has been tensilized or stretched, which decreases its diameter and makes it softer, resulting in a more flexible and slicker material than the original substrate. In addition, at each end of graft 100, pre-formed holes 2 may be present for suturing or other suitable purposes.

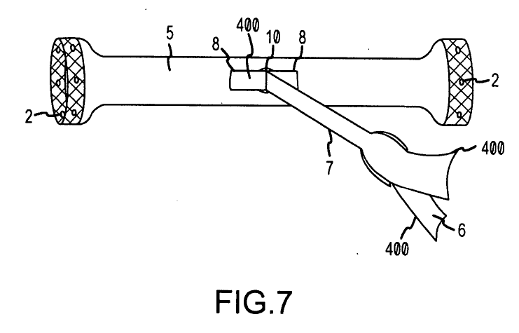

In accordance with an embodiment of the present invention, FIG. 4 illustrates “port”(s) 7 with one or more seams 410 in a lay flat position. During fabrication the seams are trimmed away from both ends of the port, forming flaps 400. In accordance with one aspect of the present invention, the seams may be trimmed by making four cuts in each end of the port. One end of this flat “tube” or port will be welded into the graft, and the other end will provide facilitated access outside the patient's body.

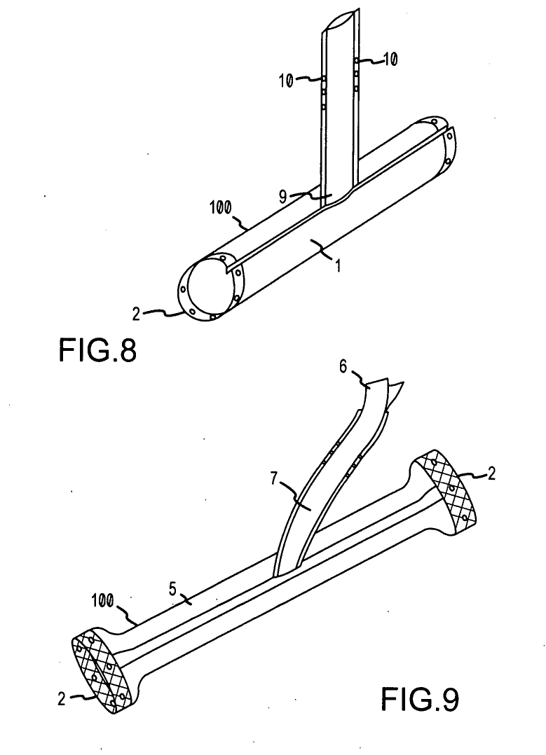

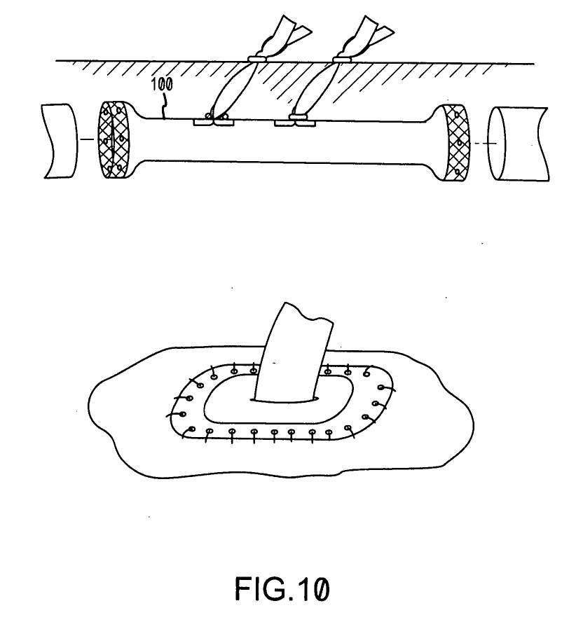

In accordance with an embodiment of the present invention, FIG. 5 illustrates port 7 after having the mid-section tensilized to make it softer, slicker, and thus more tolerable for the patient. Flaps 400 are shown at each end of port 7. With reference to FIGS. 6 and 7, port flaps 400 are welded to body 5 of vascular graft 100. FIG. 6 also illustrates the relative approximate positioning of graft 100 to a vein in the patient.

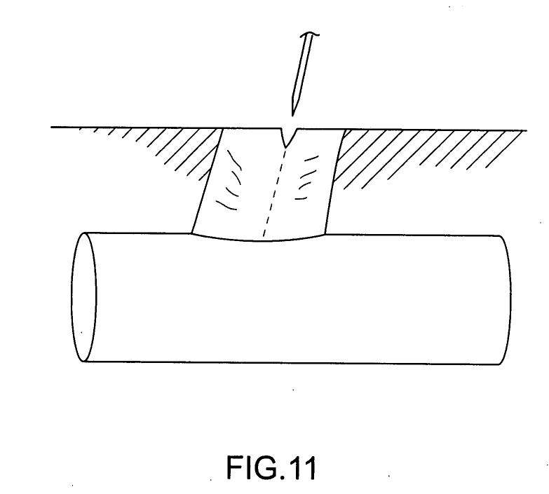

In accordance with an alternative embodiment of the present invention, FIG. 8 illustrates an alternative position for anchoring port 9 to graft 100. In this embodiment, port 9 may be positioned such that port 9 is perpendicular to the main body of graft 100. Suture anchoring holes 2 are formed at either end of graft 100. Suture holes 10 may be formed on side of port 9.

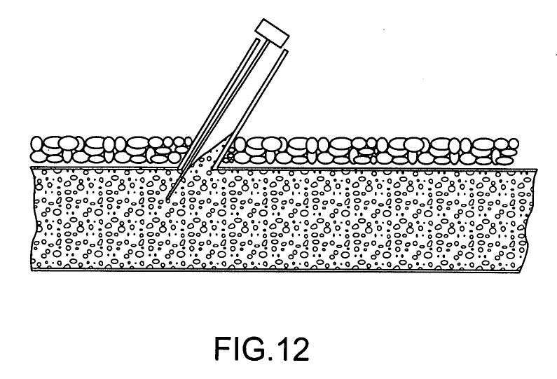

FIG. 9 illustrates port 7 as it is installed in the patient in accordance with one embodiment of the present invention. Port 7 is positioned at an approximately 45 degree angle relative to graft 100 and angled away from the direction of blood flow. This will cause the juncture of the port to the graft to close, and thus forming the first valve closest to the vessel.

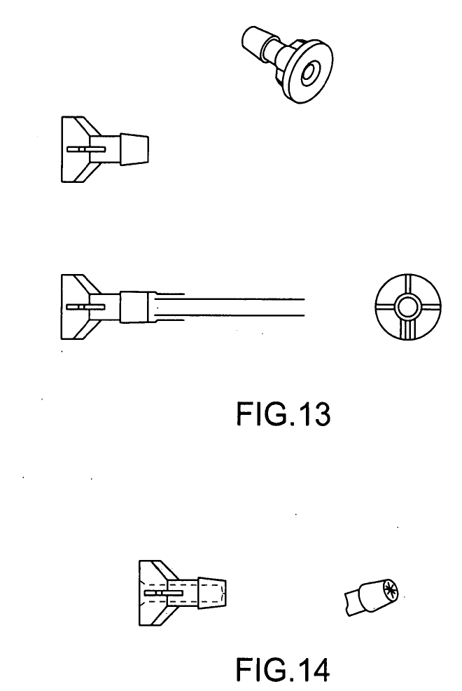

In accordance with another embodiment of the present invention, FIG. 10 illustrates an alternative design that utilizes two ports. FIG. 10 also illustrates a redundant grommet/squished O-ring that slides over the port and at that point forms another reinforced valve restriction. This valving assembly can be “positioned” by the surgeon as deemed appropriate.

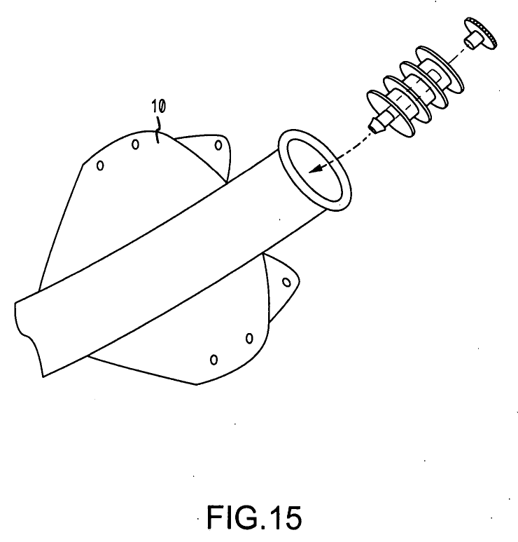

In accordance with an embodiment of the present invention, FIGS. 11 and 12 illustrate the port/graft and a cannula accessing the device.

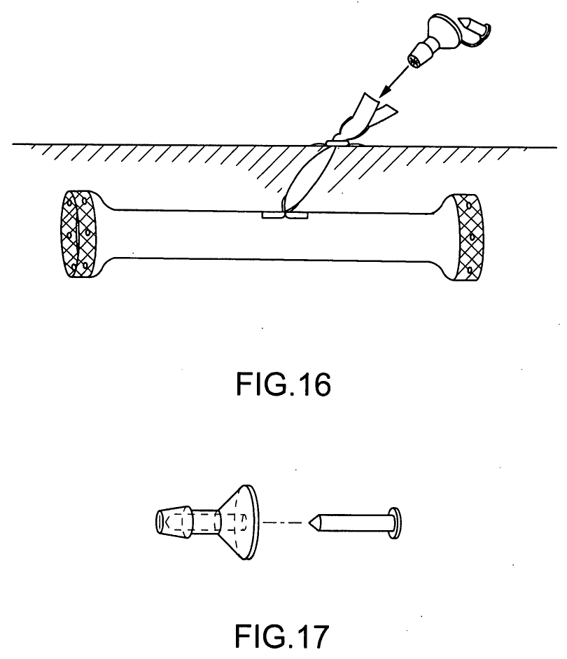

In accordance with another embodiment of the present invention, FIGS. 13-15 illustrate an entry valve that may be formed as a separate piece. The valve assembly comprises a tapered cone diaphragm which has 1-4 slots forming 2-8 sections in the diaphragm. The slots could be molded, die-cut, or laser machined (burned). The slots create a wiping action against the cannula as it is inserted and while in place during a procedure. This entry valve assembly may be suitably molded as one integral piece from materials such as urethane, nylon type 6/12, or other polymers with appropriate elastomeric flex and rigidity characteristics as determined by wall section. With reference to FIGS. 16 and 17, the valve can be hermetically sealed with a cap (snap or threaded) or a plug.

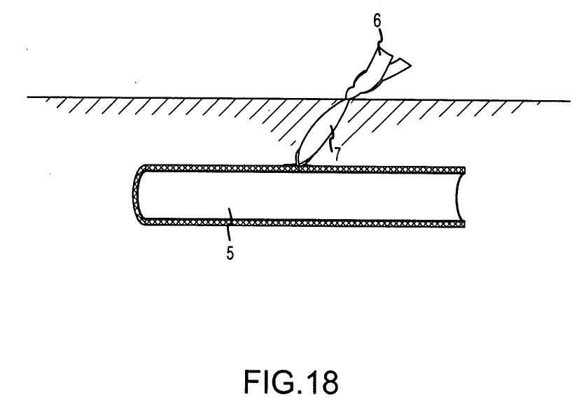

In accordance with an embodiment of the present invention, FIG. 18 illustrates the graft inside a FEP/PFA or expanded amorphous PTFE jacket. This jacket may serve in cases where additional suturing strength may be required and could be, for example, employed in a similar fashion to the port, substituting the Dacron cuff. However, all blood contact areas remain fully blood compatible film.

With reference to FIGS. 6 and 7, in accordance with an embodiment of the present invention, the vascular prosthesis 100 having a main body 5 is illustrated, generally referred to as a “vascular graft” intended to bridge a section of blood vessel or to create a shunt between different vessels and one or more secondary appendages. Ports 7 are intended to provide access from outside the patient's skin to the patient's blood vessel for the purpose of dialysis, drug infusion, nutritional supplement, and the like. An exemplary embodiment of the present invention is described for purposes of illustration, however, it should be appreciated that the invention will not be limited to this implementation. Various other uses for the graft/port system may as are now known or hereafter devised by those skilled in the art are within the scope of this invention. For example, the graft/port prosthesis may be used in other medical contexts such as a fistula for drug delivery to a specific tumor site, a vascular shunt, or a means to repeatedly draw blood samples.

When the graft/port prosthesis is used for hemomdialysis, for example, then optional suture holes 2 may be used to attach graft 100 to the artery as well as sticking through the material. Similarly, optimum suture holes 10 in the port (see FIGS. 8 and 9) may be used to anchor the port in position, either directly to body tissue or to a porous or woven cuff and in turn to body tissue. The cuff at this position may make the device more durable and robust for extended, repeated access over time.

Port 100 may act as a back flow restriction as port 7 is actually laid flat PTFE tubing, without any fixed geometry. For example, port 7 is configurable to what it is physically influenced by, such as the patient's body tissue closing it from the outside and the occasional catheter or cannula that pass through it during a procedure such that will not only restrict flow but self-purge the port of all fluids. The surgeon may close the skin exit point to a minimal size so that the lay flat tubing is bunched circumferentially allowing the patient's own muscle tissues and epidermis to close in, heal, and create further restriction to blood backflow. When the site heals and integrates, it should be somewhat naturally elastic.

In accordance with another embodiment of the present invention, a redundant polymer/elastomeric one-piece body with a valve and closure system may be integrated. The body valve closure may have a one-way diaphragm type wiper seal.

Alternatively, a grommet assembly such as that illustrated in FIGS. 10, 10A may be positioned on various locations of the port to mechanically boost back flow restrictions.

The present invention has been described above with reference to an exemplary embodiment. However, those skilled in the art will recognize that changes and modifications may be made to the exemplary embodiment without departing from the scope of the present invention. For example, the various processing steps dictated by the present invention, as well as the components for carrying out the processing steps, may be implemented in alternate ways depending upon the particular application or in consideration of any number of cost functions associated with the operation of the system. These and other changes or modifications are intended to be included within the scope of the present invention.

We claim:

1. A vascular graft for percutaneous use, the graft comprising:
a film membrane sheet formed from a fluoropolymer material, wherein the sheet forms a cylindrical shape having a first end and a second end, and wherein a seam is formed along a longitudinal direction by heat welding edges of the sheet; and
a port formed from the fluoropolymer material, wherein the port has a plurality of flaps at each end and wherein the port flaps of one end of the port are attached to the film membrane sheet.

2. The vascular graft of claim 1, wherein the film membrane sleeve comprises a polytetrafluoroethylene membrane.

3. The vascular graft of claim 1 further comprising a plurality of suture holes formed at each end of the graft.

4. The vascular graft of claim 1, where the port comprises a plurality of ports and each port is attached to the film membrane sheet by the port flaps at one end of each port.
